(19) United States
(12) Patent Application Publication
(10) Pub. No.: US 2012/0059286 A1
(43) Pub. Date: Mar. 8, 2012

(54) Self-Powered Ablation Catheter for Renal Denervation

(76) Inventors: Roger Hastings, Maple Grove, MN (US); Dave Sogard, Edina, MN (US); Kevin Edmunds, Ham Lake, MN (US); Mark L. Jenson, Greenfield, MN (US)

(21) Appl. No.: 13/227,446

(22) Filed: Sep. 7, 2011

Related U.S. Application Data
(60) Provisional application No. 61/380,422, filed on Sep. 7, 2010.
(60) Provisional application No. 61/491,728, filed on May 31, 2011.
(60) Provisional application No. 61/505,286, filed on Jul. 7, 2011.

(51) Int. Cl. A61N 7/00; A61B 18/18; A61B 18/12

(52) U.S. Cl. 601/2; 606/34; 606/33

(57) ABSTRACT

An ablation catheter includes a flexible shaft having length sufficient to access a patient's renal artery. An electrode arrangement is provided at the distal end of the shaft. A handle unit includes a housing configured for hand-held manipulation and is coupled to the catheter. A battery and one or both of a high frequency AC generator and ultrasound generator are provided in the housing. The battery serves as the sole source of power for the generator. The generator is configured to generate energy sufficient to ablate perivascular renal nerve tissue using energy stored in the battery. The catheter may be disposable and the housing re-usable. Both the catheter and the housing may be disposable.

### RELATED PATENT DOCUMENTS

This application claims the benefit of Provisional Patent Application Ser. Nos. 61/380,422 filed Sep. 7, 2010; 61/491,728 filed May 31, 2011; and 61/505,286 filed Jul. 7, 2011, to which priority is claimed pursuant to 35 U.S.C. §119(e) and which are hereby incorporated herein by reference in their entirety.

### SUMMARY

Devices, systems, and methods of the disclosure are directed to ablating target tissue of the body using a self-powered ablation catheter. Devices, systems, and methods of the disclosure are directed to denervating tissues that contribute to renal sympathetic nerve activity using high frequency AC energy delivered by a self-powered ablation catheter. Devices, systems, and methods of the disclosure are directed to denervating tissues that contribute to renal sympathetic nerve activity using ultrasound energy delivered by a self-powered ablation catheter.

Various embodiments of the disclosure are directed to ablation apparatuses and methods of ablation which include or use a self-powered ablation catheter preferably configured for hand-held manipulation. Various embodiments are directed to ablation apparatuses and methods of ablation which include or use a self-powered ablation catheter that uses a standard battery or multiple standard batteries as a sole source of power for the ablation energy source, such as a high frequency AC or an ultrasound generator. Various embodiments are directed to ablation apparatuses and methods of ablation which include or use a self-powered ablation catheter in combination with an external patient monitor.

An apparatus, according to various embodiments, includes a catheter and a handle unit coupled to the catheter. The catheter includes a flexible shaft sufficient in length to access target tissue of a patient's body. An electrode arrangement is provided at a distal end of the shaft. The handle unit includes a housing configured for hand-held manipulation. A battery is provided in the housing. A high frequency AC generator or an ultrasound generator is provided in the housing and coupled to the battery. The battery preferably serves as a sole source of power for the generator. The generator is configured to generate energy sufficient to ablate the target tissue using energy stored in the battery.

According to some embodiments, an apparatus includes a catheter having a flexible shaft sufficient in length to access a patient's renal artery. An electrode arrangement is provided at the distal end of the shaft. The apparatus further includes a handle unit comprising a housing configured for hand-held manipulation and coupled to the catheter. A battery is provided in the housing. A high frequency AC generator is provided in the housing and coupled to the battery. The generator is configured to generate energy sufficient to ablate perivascular renal nerve tissue using energy stored in the battery. The battery preferably serves as a sole source of power for the generator. The generator is configured to generate energy sufficient to ablate perivascular renal nerve tissue of at least one, and preferably both, of a patient's renal arteries.

In further embodiments, an apparatus includes a catheter having a shaft sufficient in length to access target cardiac tissue of a patient's heart. An electrode arrangement is provided at the distal end of the shaft. A handle unit includes a housing configured for hand-held manipulation and is coupled to the catheter. A battery is provided in the housing. A high frequency AC generator is provided in the housing and coupled to the battery, wherein the battery serves as a sole source of power for the generator. The generator is configured to generate energy sufficient to ablate the target cardiac tissue using energy stored in the battery.

Embodiments are directed to various methods, including a method involving supplying power using a battery provided in a hand-held self-powered handle unit of an ablation catheter device, and generating high frequency AC energy by a generator provided within the handle unit using power supplied by the battery, wherein the battery serves as a sole source of power for the generator. The method also includes communicating the high frequency AC energy to at least one electrode provided at a distal end of a catheter positioned adjacent target tissue of a patient, and ablating the target tissue using the high frequency AC energy communicated to the at least one electrode.

Other method embodiments involve supplying power using a battery provided in a hand-held self-powered handle unit of an ablation catheter device, and generating high frequency AC energy by a generator provided within the handle unit using power supplied by the battery, wherein the battery serves as a sole source of power for the generator. Such methods also involve communicating the high frequency AC energy to at least one electrode provided at a distal end of a catheter positioned within a renal artery of a patient, and ablating perivascular renal nerve tissue using the high frequency AC energy communicated to the at least one electrode.

Further method embodiments involve supplying power using a battery provided in a hand-held self-powered handle unit of an ablation catheter device, and generating high frequency AC energy by a generator provided within the handle unit using power supplied by the battery, wherein the battery serves as a sole source of power for the generator. Such methods also involve communicating the high frequency AC energy to at least one electrode provided at a distal end of a catheter positioned adjacent cardiac tissue of a patient's heart, and ablating the cardiac tissue using the high frequency AC energy communicated to the at least one electrode.

In some embodiments, a coupler is provided on the housing and adapted for connecting and disconnecting the proximal end of the catheter shaft to and from the housing, such that disposable catheters may be respectively connected and disconnected to and from the re-usable handle unit. In other embodiments, the housing comprises a battery compartment having an access panel configured to facilitate removal and replacement of the battery by a user. In further embodiments, the catheter and the handle unit are configured as disposable units.

In accordance with various embodiments, an apparatus includes a catheter comprising a flexible shaft and an ultrasound transducer provided at a distal end of the shaft. A handle unit includes a housing configured for hand-held manipulation and is coupled to the catheter. A control circuit, a battery, and a generator are respectively provided in the housing. The battery and the control circuit are coupled to the generator. The generator is coupled to the ultrasound transducer and configured to generate energy sufficient for the ultrasound transducer to ablate target tissue of the body using energy stored in the battery. The battery serves as a sole source of power for the generator.

According to some embodiments, an apparatus includes a catheter comprising a flexible shaft having a proximal end, a distal end, a length, and a lumen arrangement extending between the proximal and distal ends. The length of the shaft is sufficient to access a patient's renal artery relative to a percutaneous access location. An ultrasound transducer is provided at the distal end of the shaft. A handle unit is configured for hand-held manipulation and coupled to the catheter. A control circuit, a battery, and a generator are respectively provided in the housing. The generator is coupled to the ultrasound transducer and coupled to the battery and the control circuit. The generator is configured to generate energy sufficient for the ultrasound transducer to ablate perivascular renal nerve tissue using energy stored in the battery, the battery serving as a sole source of power for the generator.

In accordance with various embodiments, a method involves generating ultrasound energy within a hand-held ablation catheter using a battery provided in a housing of the hand-held ablation catheter. The battery serves as a sole source of power for an ultrasound generator provided in a housing of the hand-held ablation catheter. The method also involves communicating acoustic energy generated by the ultrasound generator along a catheter coupled to the handle unit and to an ultrasound transducer provided at a distal end of the catheter and positioned within or proximate target tissue of the body. The method further involves ablating the target tissue using ultrasound energy generated by the ultrasound transducer. According to some methods, the generator supplies power to the ultrasound transducer sufficient to ablate perivascular renal tissue adjacent a patient's renal nerve.

In accordance with other embodiments, a self-powered ablation catheter includes an RF ablation arrangement and an ultrasound arrangement. In some embodiments, the ultrasound arrangement is operated in a scanning or imaging mode, and the RF ablation arrangement is operated to ablate target tissue. The ultrasound arrangement, for example can be used to locate target tissue, monitor progress of the ablation by scanning the target tissue during the procedure, and/or subsequently scan the ablated tissue to verify the efficacy of the ablation. In other embodiments, the RF ablation arrangement and an ultrasound ablation arrangement of a self-powered ablation catheter can be used for ablating target tissue, and the ultrasound arrangement can also be used for scanning or imaging. For example, the different ablation arrangements can be used in tandem or individually depending on the type of target tissue and environment of use.

In some embodiments, a single transducer can be configured for both RF ablation and ultrasound ablation and/or scanning or imaging. An ultrasound transducer comprising an electrically conductive coating or element (e.g., connector or annular structure at or proximate the ultrasound transducer), for example, can serve as a combined RF ablation and ultrasound transducer. Separate generators can be housed in the handle unit of the self-powered ablation catheter. Alternatively, a single generator can be used that generates energy within a frequency range suitable for driving an RF ablation element and an ultrasound transducer.

These and other features can be understood in view of the following detailed discussion and the accompanying drawings.

### BRIEF DESCRIPTION OF THE DRAWINGS

FIG. 1 is an illustration of a right kidney and renal vasculature including a renal artery branching laterally from the abdominal aorta;

FIGS. 2A and 2B illustrate sympathetic innervation of the renal artery;

FIG. 3A illustrates various tissue layers of the wall of the renal artery;

FIGS. 3B and 3C illustrate a portion of a renal nerve;

FIG. 4 shows a system which includes a hand-held self-powered RF ablation catheter and a patient monitor in accordance with various embodiments;

FIG. 5 shows a self-powered RF ablation catheter which incorporates a cooling feature in accordance with various embodiments;

FIG. 6 shows a user interface of a self-powered ablation catheter in accordance with various embodiments;

FIG. 7 shows a self-powered ablation catheter in accordance with various embodiments;

FIG. 8 shows a self-powered ablation catheter which accommodates a guidewire in accordance with various embodiments;

FIG. 9 shows a representative schematic of ablation circuitry suitable for supplying RF energy to an electrode arrangement of a self-powered ablation catheter in accordance with various embodiments;

FIG. 10 shows a self-powered ablation catheter which incorporates an ultrasound transducer in accordance with various embodiments;

FIG. 11 shows a self-powered ablation catheter which incorporates an ultrasound transducer in accordance with other embodiments; and

FIG. 12 shows a self-powered ablation catheter which incorporates an ultrasound transducer and a flexible tether in accordance with various embodiments.

### DETAILED DESCRIPTION

Embodiments of the disclosure are directed to apparatuses and methods for ablating target tissue of the body, such as innervated tissue, cardiac tissue, organ tissue, vessels, tumors, and diseased tissue (internal and external). Embodiments of the disclosure are directed to apparatuses and methods for ablating perivascular renal nerves for the treatment of hypertension. Apparatuses and methods are directed to a self-powered ablation catheter and use of same for delivering ablation therapy to target tissue within the body.

Various embodiments of the disclosure are directed to ablation apparatuses and methods of ablation which include or use a self-powered ablation catheter preferably configured for hand-held manipulation. Various embodiments are directed to ablation apparatuses and methods of ablation which include or use a self-powered ablation catheter that uses a standard battery or multiple standard batteries as a sole source of power for the ultrasound energy source. Various embodiments are directed to ablation apparatuses and methods of ablation which include or use a self-powered ablation catheter in combination with an external patient monitor. According to some embodiments, a self-powered ablation catheter includes an RF ablation arrangement. In other embodiments, a self-powered ablation catheter includes an ultrasound ablation arrangement.

Radiofrequency ablation of renal nerves adjacent the renal artery is an emerging treatment for refractory hypertension. Conventional RF ablation systems use an ablation catheter connected to a relatively large patient-external RF generator console, which is a console similar to traditional RF ablation systems. Such conventional RF ablation systems are large, expensive, and costly to service and supply.

It has been determined by the inventors that, relative to conventional RF ablation applications such as ablation of cardiac tissue for arrhythmia treatment, renal denervation using high frequency AC energy (e.g., RF or microwave energy) has a much smaller energy requirement. According to embodiments of the disclosure, the power requirement for each renal nerve ablation typically does not exceed 8 Watts (titrated by tip temperature), which is generally applied for a maximum of two minutes. The number of lesions required in a procedure typically does not exceed eight, four in each renal artery. The total energy required for the eight ablations is significantly smaller than the typical energy required for ablation of cardiac arrhythmias. Even in higher power ablation procedures, such as those involving a maximum power of 30 Watts for up to a maximum of four minutes for each renal artery, two conventional AA lithium batteries typically provide sufficient energy for such procedures. Experimentation and analysis by the inventors has revealed that the energy requirement for renal denervation using high frequency AC energy is small enough to consider a battery operated generator contained in the handle of the ablation catheter. Various embodiments are directed to replacing relatively expensive high frequency AC generator capital equipment with relatively low-cost electronics (which may be disposable) contained entirely within the handle of the ablation catheter.

Various embodiments of a self-powered ablation catheter can provide for one or more of eliminating the need for electrical leads that cross the sterile field, no maintenance, and no service contracts. Various embodiments of a self-powered ablation catheter can provide for one or more of single operator use (no technician needed), reduced catheter lab inventory and storage space, and reduced paper work. The present disclosure sets forth computations of the energy requirement for self-powered renal nerve ablation in accordance with various embodiments, and demonstrates that conventional batteries can supply this energy for both an RF ablation arrangement and an ultrasound ablation arrangement.

According to various embodiments which incorporate an RF ablation arrangement, an efficient switching power supply is configured to operate as a representative RF generator. Similar circuitry can be implemented for self-powered renal nerve ablation devices that employ a microwave generator according to other embodiments.

In various embodiments, a cooling apparatus or mechanism is used to cool the ablation tip to spare tissues adjacent the tip from excessive heat and project heat deeper into the arterial wall to the site of the renal nerves. It is noted that various embodiments which incorporate an ultrasound ablation arrangement may not need a cooling mechanism due to the enhanced ability to focus ultrasound energy at target tissue without a thermally damaging intervening tissue. Cooling may be provided within the catheter by a circulating gas or fluid and/or by a gas phase change or Joule-Thompson effect cooling at the tip, for example. Thermocouples or other sensors can be incorporated at the ablating region of the catheter. Unipolar or bipolar electrode arrangements can be utilized. Over-the-wire, fixed-wire, or no-wire systems can be used, with guiding sheaths or catheters as needed to properly position the ablation catheter.

Various embodiments of the disclosure are directed to apparatuses and methods for renal denervation for treating hypertension. Hypertension is a chronic medical condition in which the blood pressure is elevated. Persistent hypertension is a significant risk factor associated with a variety of adverse medical conditions, including heart attacks, heart failure, arterial aneurysms, and strokes. Persistent hypertension is a leading cause of chronic renal failure. Hyperactivity of the sympathetic nervous system serving the kidneys is associated with hypertension and its progression. Deactivation of nerves in the kidneys via renal denervation can reduce blood pressure, and may be a viable treatment option for many patients with hypertension who do not respond to conventional drugs.

The kidneys are instrumental in a number of body processes, including blood filtration, regulation of fluid balance, blood pressure control, electrolyte balance, and hormone production. One primary function of the kidneys is to remove toxins, mineral salts, and water from the blood to form urine. The kidneys receive about 20-25% of cardiac output through the renal arteries that branch left and right from the abdominal aorta, entering each kidney at the concave surface of the kidneys, the renal hilum.

Blood flows into the kidneys through the renal artery and the afferent arteriole, entering the filtration portion of the kidney, the renal corpuscle. The renal corpuscle is composed of the glomerulus, a thicket of capillaries, surrounded by a fluid-filled, cup-like sac called Bowman's capsule. Solutes in the blood are filtered through the very thin capillary walls of the glomerulus due to the pressure gradient that exists between the blood in the capillaries and the fluid in the Bowman's capsule. The pressure gradient is controlled by the contraction or dilation of the arterioles. After filtration occurs, the filtered blood moves through the efferent arteriole and the peritubular capillaries, converging in the interlobular veins, and finally exiting the kidney through the renal vein.

Particles and fluid filtered from the blood move from the Bowman's capsule through a number of tubules to a collecting duct. Urine is formed in the collecting duct and then exits through the ureter and bladder. The tubules are surrounded by the peritubular capillaries (containing the filtered blood). As the filtrate moves through the tubules and toward the collecting duct, nutrients, water, and electrolytes, such as sodium and chloride, are reabsorbed into the blood.

The kidneys are innervated by the renal plexus which emanates primarily from the aorticorenal ganglion. Renal ganglia are formed by the nerves of the renal plexus as the nerves follow along the course of the renal artery and into the kidney. The renal nerves are part of the autonomic nervous system which includes sympathetic and parasympathetic components. The sympathetic nervous system is known to be the system that provides the bodies “fight or flight” response, whereas the parasympathetic nervous system provides the “rest and digest” response. Stimulation of sympathetic nerve activity triggers the sympathetic response which causes the kidneys to increase production of hormones that increase vasoconstriction and fluid retention. This process is referred to as the renin-angiotensin-aldosterone-system (RAAS) response to increased renal sympathetic nerve activity.

In response to a reduction in blood volume, the kidneys secrete renin, which stimulates the production of angiotensin. Angiotensin causes blood vessels to constrict, resulting in increased blood pressure, and also stimulates the secretion of the hormone aldosterone from the adrenal cortex. Aldosterone causes the tubules of the kidneys to increase the reabsorption of sodium and water, which increases the volume of fluid in the body and blood pressure.

Congestive heart failure (CHF) is a condition that has been linked to kidney function. CHF occurs when the heart is unable to pump blood effectively throughout the body. When blood flow drops, renal function degrades because of insufficient perfusion of the blood within the renal corpuscles. The decreased blood flow to the kidneys triggers an increase in sympathetic nervous system activity (i.e., the RAAS becomes too active) that causes the kidneys to secrete hormones that increase fluid retention and vasorestriction. Fluid retention and vasorestriction in turn increases the peripheral resistance of the circulatory system, placing an even greater load on the heart, which diminishes blood flow further. If the deterioration in cardiac and renal functioning continues, eventually the body becomes overwhelmed, and an episode of heart failure decompensation occurs, often leading to hospitalization of the patient.

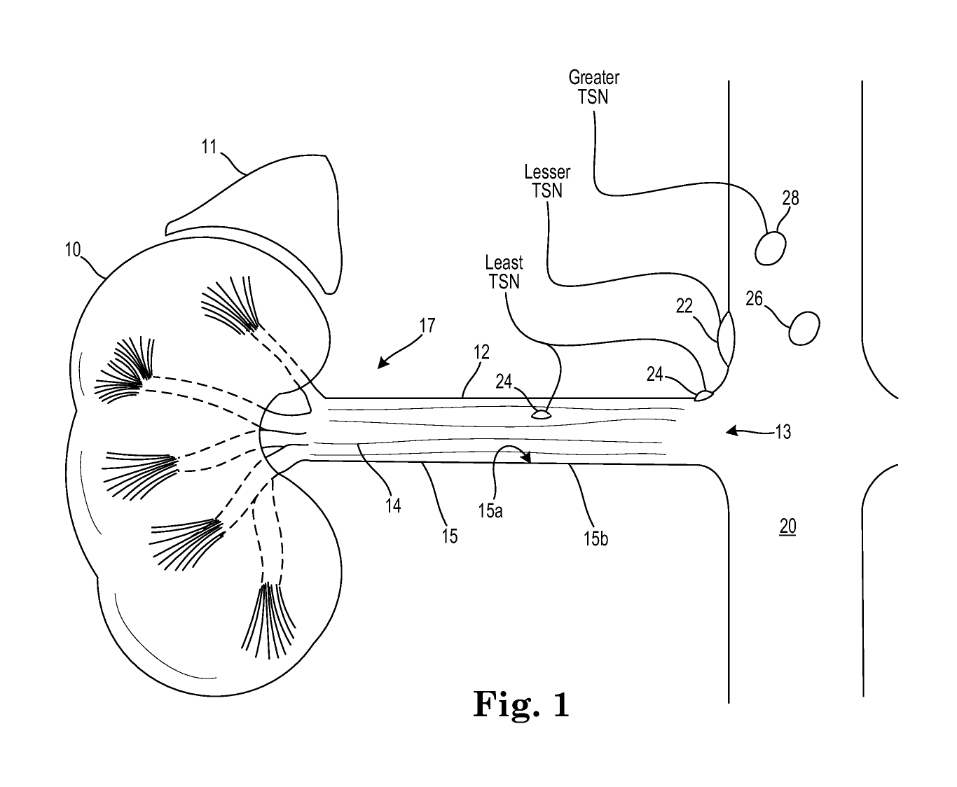

FIG. 1 is an illustration of a right kidney 10 and renal vasculature including a renal artery 12 branching laterally from the abdominal aorta 20. In FIG. 1, only the right kidney 10 is shown for purposes of simplicity of explanation, but reference will be made herein to both right and left kidneys and associated renal vasculature and nervous system structures, all of which are contemplated within the context of embodiments of the disclosure. The renal artery 12 is purposefully shown to be disproportionately larger than the right kidney 10 and abdominal aorta 20 in order to facilitate discussion of various features and embodiments of the present disclosure.

The right and left kidneys are supplied with blood from the right and left renal arteries that branch from respective right and left lateral surfaces of the abdominal aorta 20. Each of the right and left renal arteries is directed across the crus of the diaphragm, so as to form nearly a right angle with the abdominal aorta 20. The right and left renal arteries extend generally from the abdominal aorta 20 to respective renal sinuses proximate the hilum 17 of the kidneys, and branch into segmental arteries and then interlobular arteries within the kidney 10. The interlobular arteries radiate outward, penetrating the renal capsule and extending through the renal columns between the renal pyramids. Typically, the kidneys receive about 20% of total cardiac output which, for normal persons, represents about 1200 mL of blood flow through the kidneys per minute.

The primary function of the kidneys is to maintain water and electrolyte balance for the body by controlling the production and concentration of urine. In producing urine, the kidneys excrete wastes such as urea and ammonium. The kidneys also control reabsorption of glucose and amino acids, and are important in the production of hormones including vitamin D, renin and erythropoietin.

An important secondary function of the kidneys is to control metabolic homeostasis of the body. Controlling hemostatic functions include regulating electrolytes, acid-base balance, and blood pressure. For example, the kidneys are responsible for regulating blood volume and pressure by adjusting volume of water lost in the urine and releasing erythropoietin and renin, for example. The kidneys also regulate plasma ion concentrations (e.g., sodium, potassium, chloride ions, and calcium ion levels) by controlling the quantities lost in the urine and the synthesis of calcitrol. Other hemostatic functions controlled by the kidneys include stabilizing blood pH by controlling loss of hydrogen and bicarbonate ions in the urine, conserving valuable nutrients by preventing their excretion, and assisting the liver with detoxification.

Also shown in FIG. 1 is the right suprarenal gland 11, commonly referred to as the right adrenal gland. The suprarenal gland 11 is a star-shaped endocrine gland that rests on top of the kidney 10. The primary function of the suprarenal glands (left and right) is to regulate the stress response of the body through the synthesis of corticosteroids and catecholamines, including cortisol and adrenaline (epinephrine), respectively. Encompassing the kidneys 10, suprarenal glands 11, renal vessels 12, and adjacent perirenal fat is the renal fascia, e.g., Gerota's fascia, (not shown), which is a fascial pouch derived from extraperitoneal connective tissue.

The autonomic nervous system of the body controls involuntary actions of the smooth muscles in blood vessels, the digestive system, heart, and glands. The autonomic nervous system is divided into the sympathetic nervous system and the parasympathetic nervous system. In general terms, the parasympathetic nervous system prepares the body for rest by lowering heart rate, lowering blood pressure, and stimulating digestion. The sympathetic nervous system effectuates the body's fight-or-flight response by increasing heart rate, increasing blood pressure, and increasing metabolism.

In the autonomic nervous system, fibers originating from the central nervous system and extending to the various ganglia are referred to as preganglionic fibers, while those extending from the ganglia to the effector organ are referred to as postganglionic fibers. Activation of the sympathetic nervous system is effected through the release of adrenaline (epinephrine) and to a lesser extent norepinephrine from the suprarenal glands 11. This release of adrenaline is triggered by the neurotransmitter acetylcholine released from preganglionic sympathetic nerves.

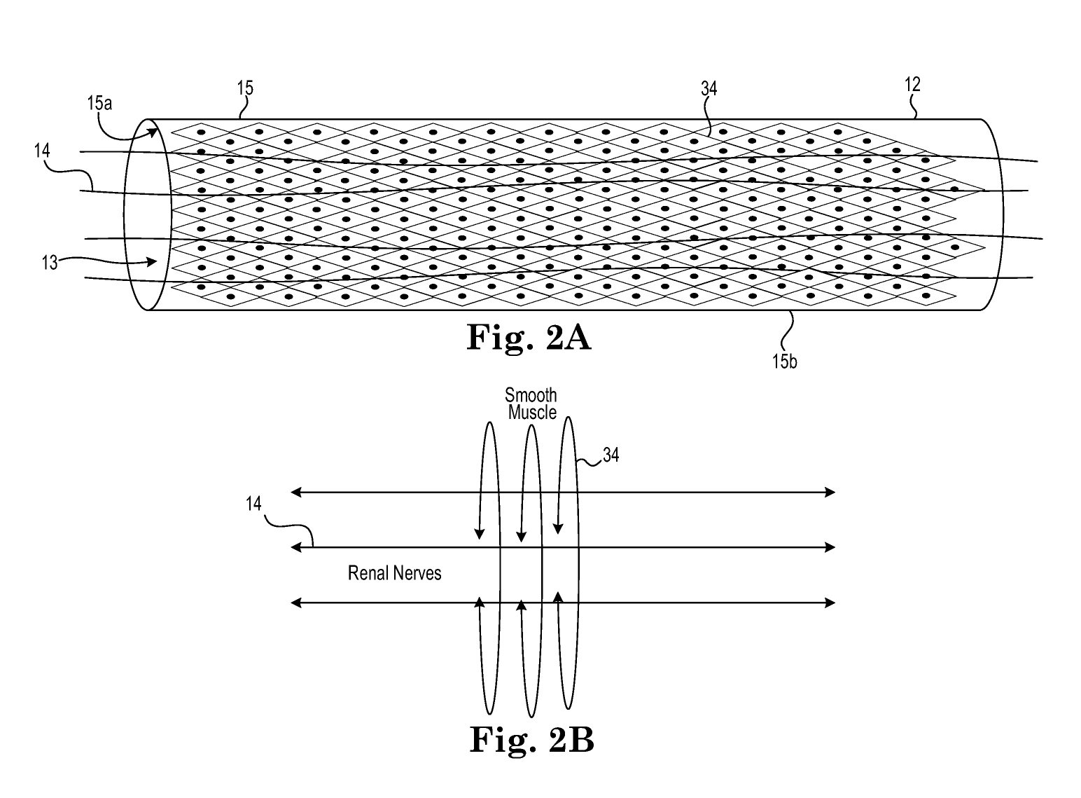

The kidneys and ureters (not shown) are innervated by the renal nerves 14. FIGS. 1 and 2A-2B illustrate sympathetic innervation of the renal vasculature, primarily innervation of the renal artery 12. The primary functions of sympathetic innervation of the renal vasculature include regulation of renal blood flow and pressure, stimulation of renin release, and direct stimulation of water and sodium ion reabsorption.

Most of the nerves innervating the renal vasculature are sympathetic postganglionic fibers arising from the superior mesenteric ganglion 26. The renal nerves 14 extend generally axially along the renal arteries 12, enter the kidneys 10 at the hilum 17, follow the branches of the renal arteries 12 within the kidney 10, and extend to individual nephrons. Other renal ganglia, such as the renal ganglia 24, superior mesenteric ganglion 26, the left and right aorticorenal ganglia 22, and celiac ganglia 28 also innervate the renal vasculature. The celiac ganglion 28 is joined by the greater thoracic splanchnic nerve (greater TSN). The aorticorenal ganglia 26 is joined by the lesser thoracic splanchnic nerve (lesser TSN) and innervates the greater part of the renal plexus.

Sympathetic signals to the kidney 10 are communicated via innervated renal vasculature that originates primarily at spinal segments T10-T12 and L1. Parasympathetic signals originate primarily at spinal segments S2-S4 and from the medulla oblongata of the lower brain. Sympathetic nerve traffic travels through the sympathetic trunk ganglia, where some may synapse, while others synapse at the aorticorenal ganglion 22 (via the lesser thoracic splanchnic nerve, i.e., lesser TSN) and the renal ganglion 24 (via the least thoracic splanchnic nerve, i.e., least TSN). The postsynaptic sympathetic signals then travel along nerves 14 of the renal artery 12 to the kidney 10. Presynaptic parasympathetic signals travel to sites near the kidney 10 before they synapse on or near the kidney 10.

With particular reference to FIG. 2A, the renal artery 12, as with most arteries and arterioles, is lined with smooth muscle 34 that controls the diameter of the renal artery lumen 13. Smooth muscle, in general, is an involuntary non-striated muscle found within the media layer of large and small arteries and veins, as well as various organs. The glomeruli of the kidneys, for example, contain a smooth muscle-like cell called the mesangial cell. Smooth muscle is fundamentally different from skeletal muscle and cardiac muscle in terms of structure, function, excitation-contraction coupling, and mechanism of contraction.

Smooth muscle cells can be stimulated to contract or relax by the autonomic nervous system, but can also react on stimuli from neighboring cells and in response to hormones and blood borne electrolytes and agents (e.g., vasodilators or vasoconstrictors). Specialized smooth muscle cells within the afferent arteriole of the juxtaglomerular apparatus of kidney 10, for example, produces renin which activates the angiotension II system.

The renal nerves 14 innervate the smooth muscle 34 of the renal artery wall 15 and extend lengthwise in a generally axial or longitudinal manner along the renal artery wall 15. The smooth muscle 34 surrounds the renal artery circumferentially, and extends lengthwise in a direction generally transverse to the longitudinal orientation of the renal nerves 14, as is depicted in FIG. 2B.

The smooth muscle 34 of the renal artery 12 is under involuntary control of the autonomic nervous system. An increase in sympathetic activity, for example, tends to contract the smooth muscle 34, which reduces the diameter of the renal artery lumen 13 and decreases blood perfusion. A decrease in sympathetic activity tends to cause the smooth muscle 34 to relax, resulting in vessel dilation and an increase in the renal artery lumen diameter and blood perfusion. Conversely, increased parasympathetic activity tends to relax the smooth muscle 34, while decreased parasympathetic activity tends to cause smooth muscle contraction.

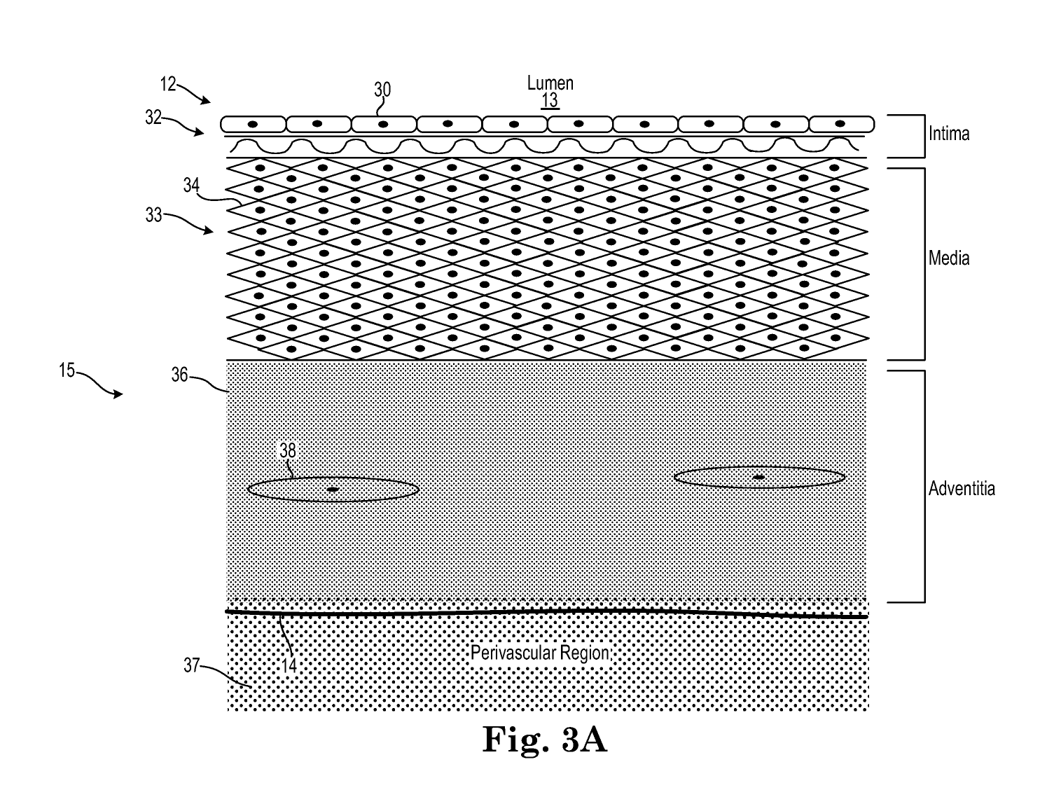

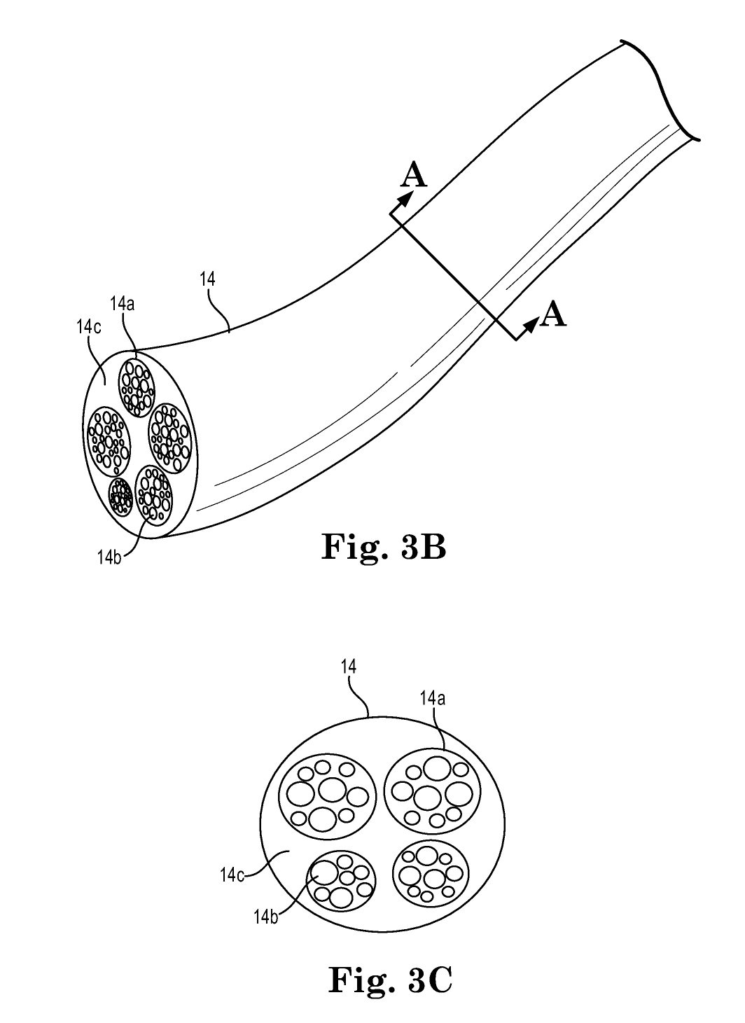

FIG. 3A shows a segment of a longitudinal cross-section through a renal artery, and illustrates various tissue layers of the wall 15 of the renal artery 12. The innermost layer of the renal artery 12 is the endothelium 30, which is the innermost layer of the intima 32 and is supported by an internal elastic membrane. The endothelium 30 is a single layer of cells that contacts the blood flowing though the vessel lumen 13. Endothelium cells are typically polygonal, oval, or fusiform, and have very distinct round or oval nuclei. Cells of the endothelium 30 are involved in several vascular functions, including control of blood pressure by way of vasoconstriction and vasodilation, blood clotting, and acting as a barrier layer between contents within the lumen 13 and surrounding tissue, such as the membrane of the intima 32 separating the intima 32 from the media 34, and the adventitia 36. The membrane or maceration of the intima 32 is a fine, transparent, colorless structure which is highly elastic, and commonly has a longitudinal corrugated pattern.

Adjacent the intima 32 is the media 33, which is the middle layer of the renal artery 12. The media is made up of smooth muscle 34 and elastic tissue. The media 33 can be readily identified by its color and by the transverse arrangement of its fibers. More particularly, the media 33 consists principally of bundles of smooth muscle fibers 34 arranged in a thin plate-like manner or lamellae and disposed circularly around the arterial wall 15. The outermost layer of the renal artery wall 15 is the adventitia 36, which is made up of connective tissue. The adventitia 36 includes fibroblast cells 38 that play an important role in wound healing.

A perivascular region 37 is shown adjacent and peripheral to the adventitia 36 of the renal artery wall 15. A renal nerve 14 is shown proximate the adventitia 36 and passing through a portion of the perivascular region 37. The renal nerve 14 is shown extending substantially longitudinally along the outer wall 15 of the renal artery 12. The main trunk of the renal nerves 14 generally lies in or on the adventitia 36 of the renal artery 12, often passing through the perivascular region 37, with certain branches coursing into the media 33 to enervate the renal artery smooth muscle 34.

Embodiments of the disclosure may be implemented to provide varying degrees of denervation therapy to innervated renal vasculature. For example, embodiments of the disclosure may provide for control of the extent and relative permanency of renal nerve impulse transmission interruption achieved by denervation therapy delivered using a treatment apparatus of the disclosure. The extent and relative permanency of renal nerve injury may be tailored to achieve a desired reduction in sympathetic nerve activity (including a partial or complete block) and to achieve a desired degree of permanency (including temporary or irreversible injury).

Returning to FIGS. 3B and 3C, the portion of the renal nerve 14 shown in FIGS. 3B and 3C includes bundles 14a of nerve fibers 14b each comprising axons or dendrites that originate or terminate on cell bodies or neurons located in ganglia or on the spinal cord, or in the brain. Supporting tissue structures 14c of the nerve 14 include the endoneurium (surrounding nerve axon fibers), perineurium (surrounds fiber groups to form a fascicle), and epineurium (binds fascicles into nerves), which serve to separate and support nerve fibers 14b and bundles 14a. In particular, the endoneurium, also referred to as the endoneurium tube or tubule, is a layer of delicate connective tissue that encloses the myelin sheath of a nerve fiber 14b within a fasciculus.

Major components of a neuron include the soma, which is the central part of the neuron that includes the nucleus, cellular extensions called dendrites, and axons, which are cable-like projections that carry nerve signals. The axon terminal contains synapses, which are specialized structures where neurotransmitter chemicals are released in order to communicate with target tissues. The axons of many neurons of the peripheral nervous system are sheathed in myelin, which is formed by a type of glial cell known as Schwann cells. The myelinating Schwann cells are wrapped around the axon, leaving the axolemma relatively uncovered at regularly spaced nodes, called nodes of Ranvier. Myelination of axons enables an especially rapid mode of electrical impulse propagation called saltation.

In some embodiments, a treatment apparatus of the disclosure may be implemented to deliver denervation therapy that causes transient and reversible injury to renal nerve fibers 14b. In other embodiments, a treatment apparatus of the disclosure may be implemented to deliver denervation therapy that causes more severe injury to renal nerve fibers 14b, which may be reversible if the therapy is terminated in a timely manner. In preferred embodiments, a treatment apparatus of the disclosure may be implemented to deliver denervation therapy that causes severe and irreversible injury to renal nerve fibers 14b, resulting in permanent cessation of renal sympathetic nerve activity. For example, a treatment apparatus may be implemented to deliver a denervation therapy that disrupts nerve fiber morphology to a degree sufficient to physically separate the endoneurium tube of the nerve fiber 14b, which can prevent regeneration and re-innervation processes.

By way of example, and in accordance with Seddon's classification as is known in the art, a treatment apparatus of the disclosure may be implemented to deliver a denervation therapy that interrupts conduction of nerve impulses along the renal nerve fibers 14b by imparting damage to the renal nerve fibers 14b consistent with neruapraxia. Neurapraxia describes nerve damage in which there is no disruption of the nerve fiber 14b or its sheath. In this case, there is an interruption in conduction of the nerve impulse down the nerve fiber, with recovery taking place within hours to months without true regeneration, as Wallerian degeneration does not occur. Wallerian degeneration refers to a process in which the part of the axon separated from the neuron's cell nucleus degenerates. This process is also known as anterograde degeneration. Neurapraxia is the mildest form of nerve injury that may be imparted to renal nerve fibers 14b by use of a treatment apparatus according to embodiments of the disclosure.

A treatment apparatus may be implemented to interrupt conduction of nerve impulses along the renal nerve fibers 14b by imparting damage to the renal nerve fibers consistent with axonotmesis. Axonotmesis involves loss of the relative continuity of the axon of a nerve fiber and its covering of myelin, but preservation of the connective tissue framework of the nerve fiber. In this case, the encapsulating support tissue 14c of the nerve fiber 14b are preserved. Because axonal continuity is lost, Wallerian degeneration occurs. Recovery from axonotmesis occurs only through regeneration of the axons, a process requiring time on the order of several weeks or months. Electrically, the nerve fiber 14b shows rapid and complete degeneration. Regeneration and re-innervation may occur as long as the endoneural tubes are intact.

A treatment apparatus may be implemented to interrupt conduction of nerve impulses along the renal nerve fibers 14b by imparting damage to the renal nerve fibers 14b consistent with neurotmesis. Neurotmesis, according to Seddon's classification, is the most serious nerve injury in the scheme. In this type of injury, both the nerve fiber 14b and the nerve sheath are disrupted. While partial recovery may occur, complete recovery is not possible. Neurotmesis involves loss of continuity of the axon and the encapsulating connective tissue 14c, resulting in a complete loss of autonomic function, in the case of renal nerve fibers 14b. If the nerve fiber 14b has been completely divided, axonal regeneration causes a neuroma to form in the proximal stump.

A more stratified classification of neurotmesis nerve damage may be found by reference to the Sunderland System as is known in the art. The Sunderland System defines five degrees of nerve damage, the first two of which correspond closely with neurapraxia and axonotmesis of Seddon's classification. The latter three Sunderland System classifications describe different levels of neurotmesis nerve damage.

The first and second degrees of nerve injury in the Sunderland system are analogous to Seddon's neurapraxia and axonotmesis, respectively. Third degree nerve injury, according to the Sunderland System, involves disruption of the endoneurium, with the epineurium and perineurium remaining intact. Recovery may range from poor to complete depending on the degree of intrafascicular fibrosis. A fourth degree nerve injury involves interruption of all neural and supporting elements, with the epineurium remaining intact. The nerve is usually enlarged. Fifth degree nerve injury involves complete transection of the nerve fiber 14b with loss of continuity.

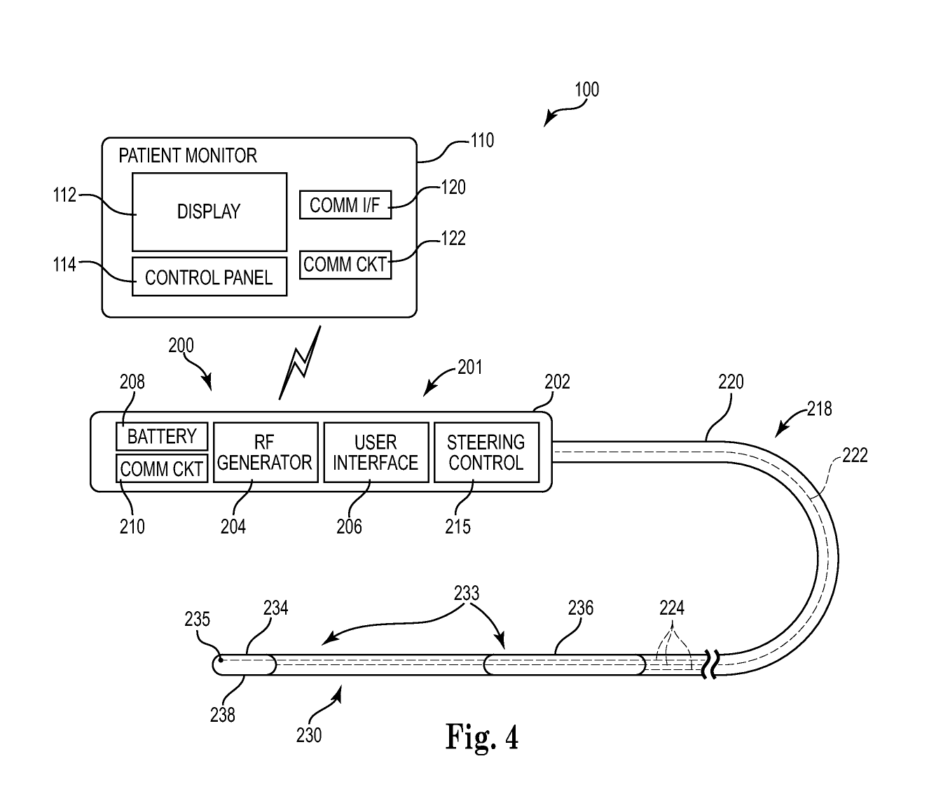

Referring to FIG. 4, there is shown a medical system 100 which includes a self-powered ablation catheter 200 and a patient monitor 110 in accordance with various embodiments. The self-powered ablation catheter 200 includes a housing 201 which is configured as a handle unit 202 for hand-held manipulation by a clinician. The housing 201 includes a number of components including an RF generator 204 coupled to a battery 208. The RF generator 204 is configured to generate energy sufficient to achieve renal denervation using energy stored in the battery 208. The battery 208, as discussed herein in greater detail, preferably includes one or a number of conventional, readily available batteries. The batteries are preferably disposable. The battery 208 preferably serves as the sole source of power for at least the RF generator 204. It is preferable that the battery 208 serves as the sole source of power for all components of the ablation catheter 200.

The housing 201 supports a user interface 206 which includes a number of switches and one or more displays that facilitate control of the self-powered ablation catheter 200 by a clinician. A steering control 215 is also included in or on the housing 201 in accordance with the embodiment shown in FIG. 4. The steering control 215 is intended to represent various known steering mechanisms that allow the clinician to direct a proximal end 230 of the catheter 218 to a target location, such as a patient's renal artery.

In some embodiments, the housing 201 may include communication circuitry 210 configured to effect communications with a communications circuit 122 of a patient monitor 110 or other device. The patient monitor 110 shown in FIG. 4 includes a display 112 and a control panel 114 comprising a variety of controls and switches. Although the patient monitor 110 is an optional device, the display 112, memory (not shown), and other features of the patient monitor 110 may provide for enhanced feedback and information useful to the clinician. It is to be understood that the self-powered ablation catheter 200 can be used to perform ablation procedures without need of the patient monitor 110 or other device.

Data stored within and/or communicated from the ablation catheter 200 preferably includes one or more of ablation start and stop time, number of ablations performed, battery life remaining, temperature, impedance and power versus time during the ablation, etc. This data can also include RF voltage and current amplitudes (and temperature) during the ablation. In some embodiments, the communication circuitry 210 can be configured for two-way communication. In other embodiments, the communication circuitry 210 can be configured for one-way communication.

The patient monitor 110 may be communicatively coupled to a patient information management system via a communications interface 120, and can transfer data received from the ablation catheter 200 into the patient's medical record. The data may also be displayed on a patient monitor 110 via display 112, for example as temperature and power versus time. Other parameters and patient-related information described herein may be displayed.

As is further shown in FIG. 4, the catheter 218 is coupled to the handle unit 202. The catheter 218 includes a flexible shaft 220 having a proximal and, a distal end 230, and a lumen arrangement to 222 extending between the proximal and distal ends. The length of the shaft 220 is sufficient to access a patient's renal artery from a percutaneous location. One or more electrical conductors 224 extend along the shaft 220 preferably within the lumen arrangement 222. An electrode arrangement 233 is provided at the distal end 230 of the shaft 220 and is coupled to the electrical conductor arrangement 224.

In some embodiments, the catheter 218 is detachably coupled to the handle unit 202 via a coupler, allowing for replacement of the catheter 218 following an ablation procedure and re-use of the handle unit 202. The coupler facilitates both mechanical, fluidic (optional), and electrical coupling between the catheter shaft 220, lumen arrangement 222, and electrical conductors 224. In other embodiments, the catheter 218 is permanently connected to the handle unit 202, such that the entire ablation catheter 200 is disposable.

In some embodiments, the electrode arrangement 233 includes at least two electrodes 234, 236 which are operated in a bipolar mode. In a bipolar configuration, it is preferable that the return electrode 236 be significantly larger in surface area than the ablation tip electrode 234 in order to prevent or reduce heating adjacent the return electrode. In other embodiments, a single ablation electrode 234 can be used together with an external electrode for operating in a unipolar mode. It may be preferable for the self-powered ablation catheter 200 to operate in a bipolar mode so that an external return electrode is not needed.

In FIG. 4, the electrode arrangement 233 at the distal end 230 of the shaft 220 includes a pair of electrodes 234, 236 arranged in a spaced-apart relationship. The ablation electrode 234 of the electrode pair is preferably situated near the tip 238 of the shaft 220. The return electrode 236 is preferably spaced between about 30 mm and 300 mm from the distal electrode 234. The electrodes 234 and 236 preferably have a diameter between about 1 mm and 2 mm. The ablation electrode 234 preferably has a length between about 1 mm and 4 mm and the return electrode 236 preferably has a length between about 4 mm and 50 mm. The tip 238 of the catheter shaft 220 is preferably constructed as a flexible atraumatic tip.

A temperature sensor 235 is shown positioned at or proximate the ablation electrode 234. The temperature sensor 235 is used to measure the temperature at the artery wall adjacent to the ablation electrode 234. One or more additional temperature sensors 235 may be included, such as a proximal temperature sensor 235 at or proximate the return electrode 236 if desired. Temperature signals provided by the one or more temperature sensors 235 are preferably communicated to a processor disposed in the housing 201 of the self-powered ablation catheter 200. The temperature sensor information may be used to automatically adjust the energy generated by the RF generator 204 to maintain appropriate tissue temperatures during ablation.

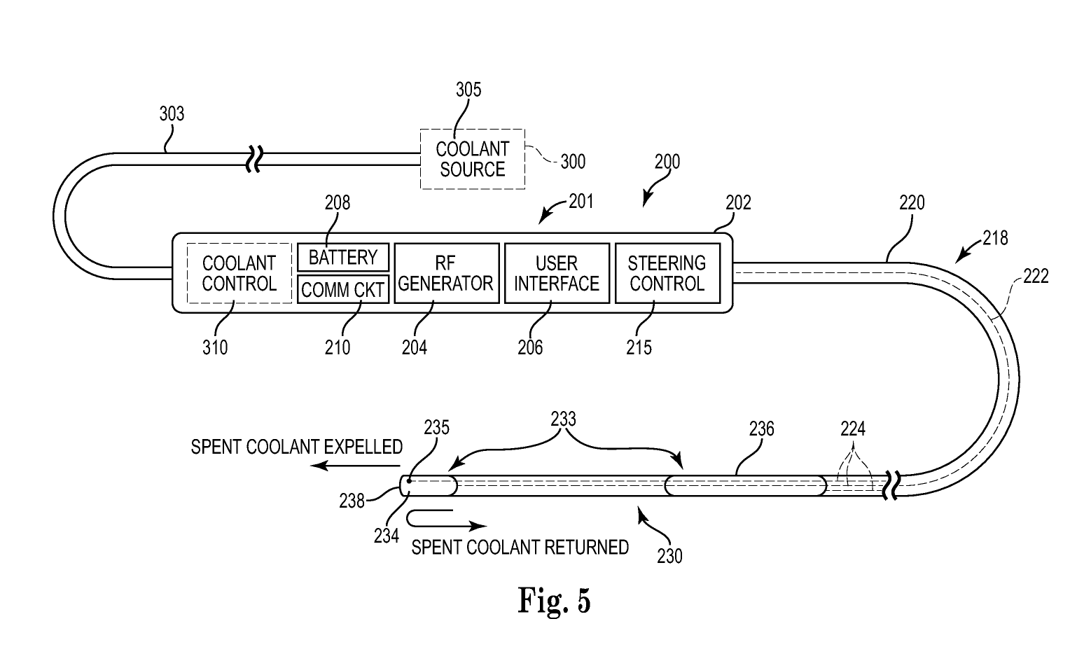

Referring to FIG. 5, embodiments of a self-powered ablation catheter 200 are shown which incorporate a cooling feature. According to FIG. 5, the housing 201 of the self-powered ablation catheter 200 includes a coolant control 310 which provides for clinician control over the delivery of coolant 305 from an external coolant source 300 to the distal end 230 of the shaft 220. According to some implementations, the handle unit 202 includes a coolant lumen fluidly coupled to the lumen arrangement 222 of the catheter shaft 220 and a supply tube 303 fluidly coupled to the coolant source 300. The coolant control 310 includes one or more controls that allow for clinician adjustment of coolant dispensing rate and coolant temperature. The coolant source 300 typically includes a reservoir fluidly coupled to a pump. A cooling agent 305 is contained within the reservoir. In a simple embodiment, the cooling agent is an elevated bag of sterile saline, and the pumping means is gravity. Saline at room temperature is cool relative to body temperature.

In some embodiments, the cooling arrangement of the self-powered ablation catheter 200 is a closed system in which spent coolant 305 is returned from the distal end 230 of the shaft 220 to the coolant source 300. A variety of coolant may be employed in a closed cooling arrangement, including cold saline or cold saline and ethanol mixture, Freon, or other fluorocarbon refrigerants, nitrous oxide, liquid nitrogen, and liquid carbon dioxide, for example. In some embodiments, the cooling agent 305, when released inside a cooling chamber at the distal end 230 of the catheter shaft 220, undergoes a phase change that cools the distal end 230 of the catheter shaft 220, such as by the Joule-Thomson effect.

In other embodiments, a biocompatible cooling agent is used as the coolant 305, allowing for spent coolant 305 to be expelled from the distal end 230 of the catheter shaft 220 through an exit port arrangement. Suitable coolants for an open cooling arrangement include cold sterile saline, Ringer's solution or other blood compatible fluids. Inclusion of one or more temperature sensors 235 at or proximate one or both of electrodes 234 and 236 in the embodiments shown in FIG. 5 allows for automatic delivery and adjustment of RF energy and coolant during renal denervation.

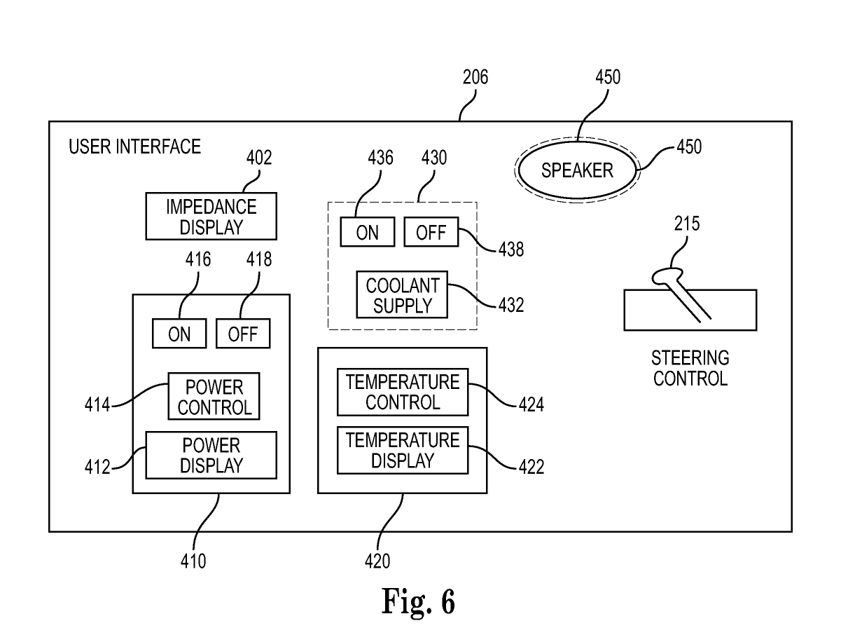

FIG. 6 shows a user interface 206 of a hand-held self-powered ablation catheter 200 in accordance with embodiments of the disclosure. The user-interface 206 includes a power section 410, a temperature section 420, an optional cooling section 430, a steering control section 215, and an optional audio output section 450. The power section 410 is shown to include a power control 414, a power ON switch 416 a power OFF switch 418, and a display 412. An impedance display 402 may also be provided to indicate actual impedance values or an indication (e.g., color) that tissue impedance is within or outside of a predefined impedance range.

The temperature control section 420 is shown to include a temperature control 424 and a temperature display 422. The cooling section 430 is shown to include an ON switch 436, an OFF switch 430, and a coolant supply control 432. The audio output section 450 includes a speaker 455 and may additionally include a microphone. The microphone may be used to record comments made by the clinician during an ablation procedure. The microphone may also be used to implement voice-activated commands issued by the clinician, such as one or more of power, temperature, coolant delivery, and steering commands. Other display features may include one or more indicator lights for power ON and OFF or a fault condition. A timer may display ablation time elapsed, or may count down a preset ablation duration.

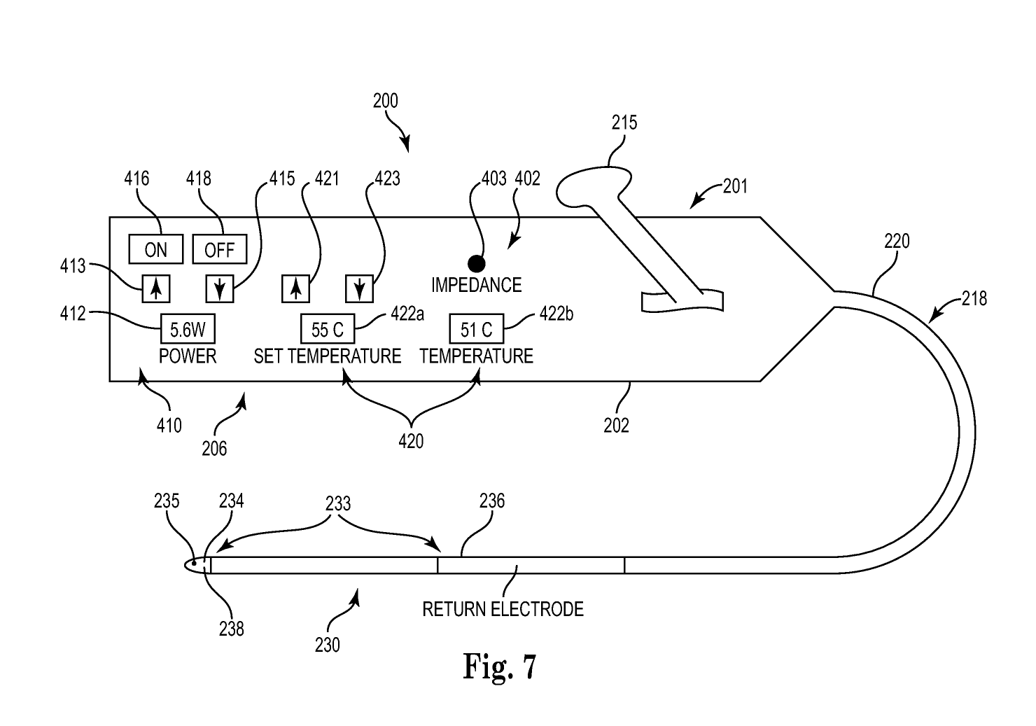

FIG. 7 shows a hand-held self-powered ablation catheter 200 in accordance with various embodiments of the disclosure. In the embodiment shown in FIG. 7, a number of switches and displays are provided on the housing 201 of the ablation catheter 200. Controlling power states of ON/OFF and UP/DOWN (in terms of Watts) is preferably controlled by soft keys on the handle housing 201, and displayed on one or more display screens incorporated into the handle housing 201. For example, and as shown in FIG. 7, the power section 410 includes a power increase switch 413 (UP increment switch), a power decrease switch 415 (DOWN increment switch), an ON switch 416, and an OFF switch 418. The power section 410 further includes a power display 412, which shows 5.6 W in the representative illustration of FIG. 7.

The temperature section 420 similarly includes a temperature increase switch 421 (UP increment switch) and a temperature decrease switch 423 (DOWN increment switch). The temperature section 420 includes to temperature displays 422a and 422b. Temperature display 422a shows the temperature set by the clinician, which is shown as 55° C. in this illustrative embodiment. The actual temperature as sensed by a temperature sensor 235 at the electrode arrangement 233 of the shaft 220 is shown in temperature display 422b, which shows a temperature of 51° C. in this illustrative embodiment. The impedance display 402 in this embodiment includes an impedance indicator 403. The impedance indicator 403 preferably indicates that the impedance is “in range” in green and “out of range” in red. It is understood that other colors and indications can be used to indicate the status of tissue impedance.

The handle unit 202 further includes a steering control 215 which allows the clinician to steer the shaft 220 of the catheter 218. Deflection of the tip 238 of the catheter shaft 220 can be controlled, for example, using a steerable catheter mechanism, such as a mechanism similar to those used in electrophysiology (EP) catheters.

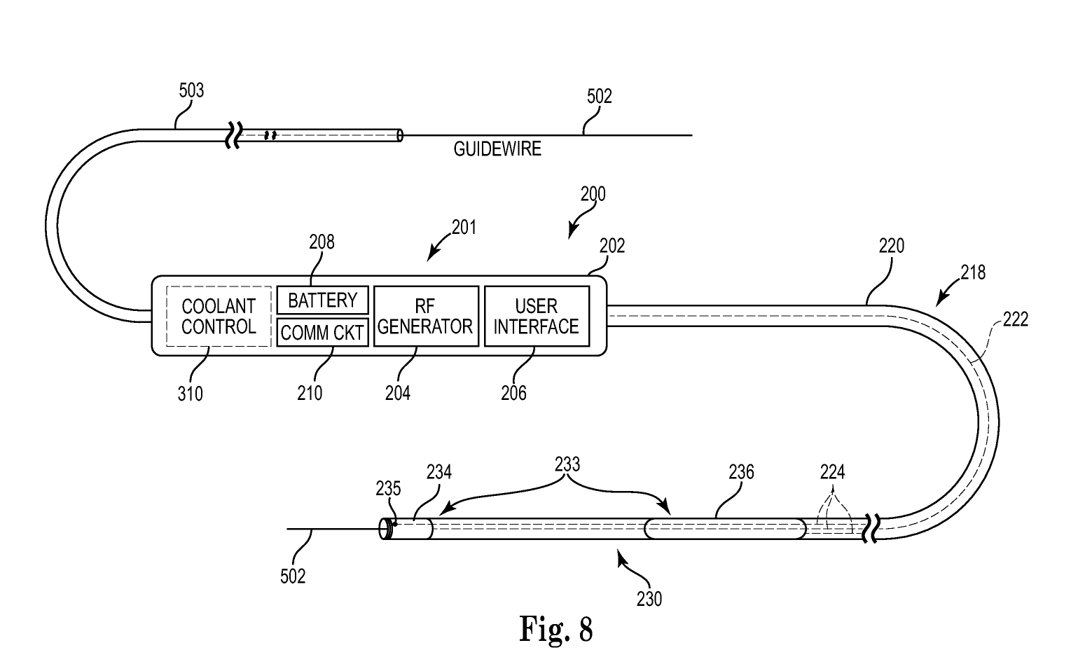

FIG. 8 shows a self-powered ablation catheter 200 in accordance with various embodiments of the disclosure. In the embodiment shown in FIG. 8, a guidewire 502 is employed for purposes of advancing the catheter shaft 220 to the target location, such as a patient's renal artery. The handle 202, in this embodiment, includes a guidewire lumen or channel coupled to a guide lumen of the lumen arrangement 222 of the catheter shaft 220. A guide tube 503 can be connected to the proximal end of the handle 202 to facilitate easy advancement and retraction of the guidewire 502 to and from the handle 202. Various known over-the-wire techniques may be used to advance the catheter shaft 222 the patient's renal artery. It is noted that the lumen arrangement 222 may include either an open or closed coolant dispensing/circulation apparatus for embodiments which provide cooling at the artery wall.

According to various embodiments, such as those illustrated in FIGS. 4-8, all or particular components of the self-powered ablation catheter 200 are preferably implemented to be disposable. In some embodiments, the entire ablation catheter device 200 is implemented to be disposable. In other embodiments, the handle unit 202 is implemented to be re-usable, while the catheter section 220 is implemented to be disposable.

In their experimentation/analysis, the inventors considered the energy required to ablate at four points in each of two renal arteries, using a maximum power of 8 Watts for a maximum time of 2 minutes. The energy required for a single ablation is 8 Watts times 120 seconds, or 960 Joules. The total energy required to create eight lesions at the maximum power and time settings is then 7,680 Joules.

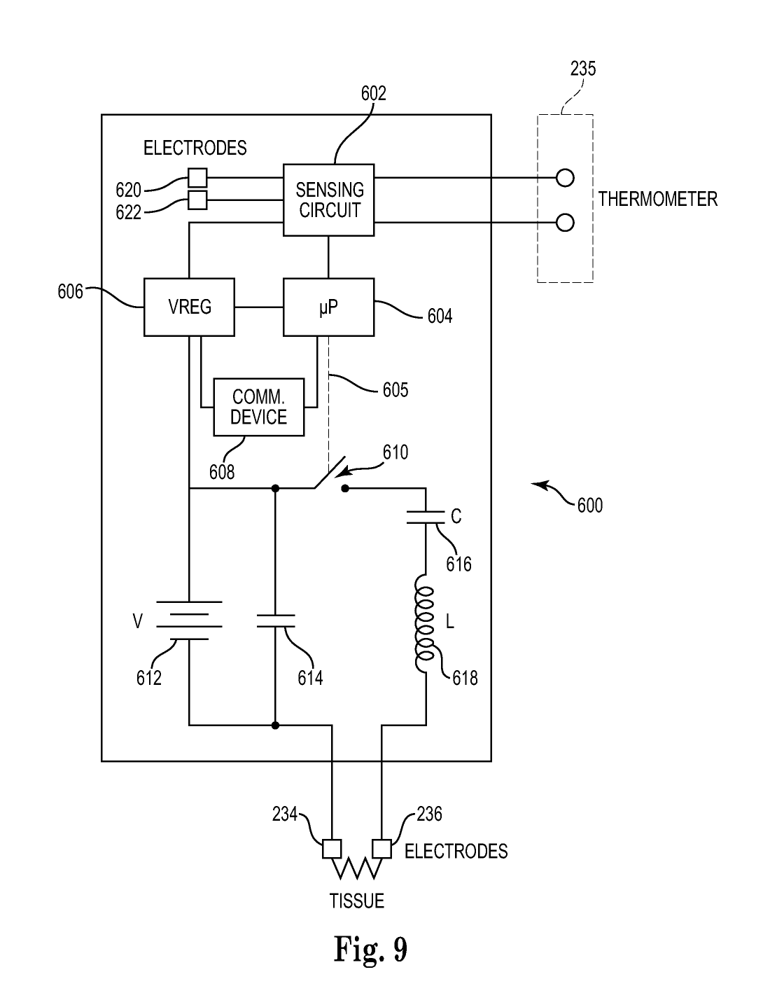

The energy consumed by the ablation electronics of the self-powered ablation catheter 200 (FIG. 9) would likely exceed this value, but modem switching power supplies are very efficient, and use a fraction of this energy, and importantly do not heat up the handle unit 202. Measurement and display electronics use minimal power. To be conservative, the ablation energy requirement was multiplied by two, for a power requirement of about 15,000 Joules.

A single AA alkaline battery can supply more than 12,000 Joules, while a lithium AA can supply twice this energy (about 24,000 Joules). Thus, two alkaline or one lithium AA battery can supply the energy needed for a renal denervation therapy procedure in accordance with apparatuses and methods of the present disclosure.

In accordance with some embodiments, a higher power renal artery denervation procedure may involve performing about 4 to 6 ablations in each renal artery (repositioning the RF electrode each time). Assuming the energy required for a single ablation is 8 Watts times 120 seconds (2 minutes), or 960 Joules, the total energy required to create between eight and twelve lesions at the maximum power and time settings ranges between about 7,680 to about 11,520 Joules. To be conservative, the ablation energy requirement for this representative example can be multiplied by two, for a power requirement ranging between about 15,000 and 23,000 Joules. The capacity of one AA lithium battery or two AA alkaline batteries can supply the energy needed for this higher power procedure.

According to other embodiments, it may be desirable to perform RF ablation of perivascular renal nerve tissue at higher power and for longer durations but without needing multiple ablations in each renal artery. Such higher power renal artery denervation procedures may require a capacity of between about 12 to 24 Watts (and possibly as high as about 30 Watts) for up to about 4 minutes for each artery. The total energy required for this representative higher power renal artery denervation procedure (i.e., using 12 to 30 Watts for up to 4 minutes for each artery) ranges between about 5,760 to about 14,400 Joules. To be conservative, the ablation energy requirement for this representative example can be multiplied by two, for a power requirement ranging between about 11,500 and 29,000 Joules. The capacity of two lithium AA batteries (about 48,000 Joules) can supply the energy needed for this higher power procedure.

It is understood that some embodiments of a self-powered ablation catheter may be implemented to house larger batteries (e.g., larger than AA batteries, such as C or D batteries) and/or greater than two batteries (e.g., 3 or 4 AA batteries) depending on the power requirements of a particular ablation catheter design. In such embodiments, the housing of the self-powered ablation catheter can be made larger to accommodate larger and/or more numerous batteries. However, the handle unit of the self-powered ablation catheter should remain ergonomically efficient, so as not to unduly limit a clinician's ability to manipulate the ablation catheter during the time period required to perform ablations in each of a patient's two renal arteries.

The RF voltage amplitude required to ablate at an average power of 8 Watts may be estimated from a typical value of tissue resistance. The Ohmic heat generated in the tissue is given by V²/2R. Setting this equal to 8 Watts, and using a typical tissue resistance of 100 Ohms, yields a voltage amplitude of 40 Volts. The current amplitude is equal to V/R or 0.4 amps.

In various embodiments, the switching power supply can be powered by two 40 Volt batteries (+/−40 Volts), which may consist of, for example, four A23 12 volt batteries in series, yielding a 48 volt battery. Three stacks of these button batteries in parallel is roughly the volume of a AA battery. This battery pack will readily supply the 0.4 amp amplitude, or 280 mA RMS current required.

Referring to FIG. 9, there is shown a representative schematic of ablation circuitry 600 of a self-powered ablation catheter 200 according to various embodiments. The ablation circuitry 600 includes a switching power supply suitable for supplying ablation power to ablation electrodes 234 and 236. The circuitry 600 shown in FIG. 9 is small enough to fit within the handle unit 202 of the ablation catheter 200. The battery 612 supplies DC voltage that is converted to pulsed DC voltage by turning switch 610 on and off. The capacitor 616 block DC voltage from the electrodes 234 and 236, and with the smoothing action of the LC components, converts the pulsed DC voltage to sine wave voltage having one half the amplitude of battery 612. According to a representative example, battery 612 can consists of two 40 Volt batteries connected in series, or one 80 Volt battery. Other circuits can be employed that switch between two 40 Volt batteries to create a +/−40 Volt sine wave.

In a representative mode of operation, a microprocessor 604 switches on and off at a desired RF ablation frequency, e.g. 480 kHz. The LC circuit 618, 616 converts the on/off square waves to a sine wave that is delivered to the tissue electrodes 234 and 236 for ablation. Ablation occurs only around the tip electrode 236 because it has a sufficiently small area to create a current density large enough to elevate tissue temperature. The power delivered to the tissue is controlled in some embodiments by delivering bursts of RF power, and adjusting the time off between bursts (duty cycle modulation). The duty cycle can be either the off time of individual 480 kHz cycles or the off time of bursts that consist of a number of 480 kHz cycles.

Feedback from an ablation tip thermometer 235 may be fed back to the microprocessor 604 to automatically control tip temperature. Feedback from the catheter electrodes 620 and 622 measures tissue impedance, used, for example, to shut power off if an impedance rise relative to (e.g., and exceeding) a threshold is sensed. One or both of electrodes 620 and 622 may be the same as electrodes 234 and 236. The tissue voltage is also measured and multiplied by measured RF current and averaged to compute RF power delivered to the tissue, which is adjusted up until a set temperature or set power is reached.

The power supply 612 shown in FIG. 9 (e.g., 2 40 Volt batteries in series or one 80 Volt battery) may supply a peak RF voltage of about +/−40 volts, or a boost regulator (VREG) 606, may boost the battery voltage, for example, from about 3 to about 80 volts. Capacitor 614 is charged when switch 610 is open, and provides rapid current flow when switch 610 is closed, thereby preventing a sag or drop in battery voltage 612. The microprocessor 604 controls an efficient switch 610, e.g., a FET, to deliver on and off voltage pulses to the tissue electrodes 234 and 236. The inductor 618 and capacitor 616 form a tank circuit that is tuned to the switching frequency, and filter the square waves to form a sine wave output at the electrodes 234 and 236.

A typical RF ablation frequency is 480 kHz. Tissue power is controlled, for example, by adjusting the duty cycle of RF energy delivered to the electrodes 234 and 236. Signals from the tip thermometer 235 and tissue impedance measured at the electrodes 620 and 622 are fed back to the microprocessor 604 to automatically control power, e.g., duty cycle, to maintain a constant tip temperature, and to shut down power if the impedance rises above a set limit.

In various embodiments, a conventional return electrode pad can be used instead of the catheter borne return electrode shown in FIG. 4, for example. The pad would be unpacked in the sterile field, attached to the patient, and plugged into the handle unit 202 of the ablation catheter 200.

In general, when renal artery tissue temperatures rise above about 113° F. (50° C.), protein is permanently damaged (including those of renal nerve fibers). If heated over about 65° C., collagen denatures and tissue shrinks. If heated over about 65° C. and up to 100° C., cell walls break and oil separates from water. Above about 100° C., tissue desiccates. According to some embodiments, the ablation circuitry 600 is configured to maintain the current densities at the ablation electrode 234 at a level sufficient to cause heating of the target tissue preferably to a temperature of at least 55° C.

A preferred ablation catheter embodiment would spare the arterial smooth muscle tissues adjacent the ablation electrode, while ablating the renal nerves adjacent the outside of the arterial wall (i.e., perivascular renal nerves and ganglia). This can be accomplished by cooling the ablation tip in a manner described previously, while current that penetrates beyond the cooling zone is still capable of heating and ablating the nerves. In some embodiments, a miniaturized version of a cooling or cryogenic catheter system (e.g., balloon catheter) may be employed, with a significantly reduced cooling volume. Other cooling apparatuses and mechanisms may be employed, including circulating a cooled liquid or gas, converting a liquid to a gas at the ablation tip, and/or passing a gas through a nozzle within the tip to cool via the Joule-Thompson effect. In some embodiments a miniature Peltier effect solid state cooler may be incorporated into tip electrode 234.

As was discussed previously, the handle unit 202 of the ablation catheter 200 can include a communications facility. In the schematic diagram of FIG. 9, for example, a communications device 608 is shown powered by the voltage regulator 606 and controlled by the microprocessor 604, although other configurations are contemplated. The communications device 608 can be configured for either bi-directional or uni-directional wireless and/or wired communication with a patient monitor or other external system.

For example, the communications device 608 may implement a wireless communications protocol, such as Bluetooth or Zigbee, for example. Other suitable wireless protocols include Medical Implant Communication Service (MICS), Industrial, Scientific and Medical (ISM), and Short Range Devices (SRD) protocols, among others. In some embodiments, a wired connection between the self-powered ablation catheter 200 and patient monitor 110 (or other device or system) can be used as a primary communication link or a secondary/backup communication link (e.g., secondary/backup to a primary wireless link). Suitable wired communication protocols include Wired Ethernet (IEEE 802.3), FireWire™, and USB protocols, among others. In some hybrid embodiments, power from a USB cable may be used together with, or to the exclusion of, battery 208. In some embodiments, for example, a USB cable may be used to recharge a standard rechargeable battery 208 (e.g., lithium ion battery) of a self-powered ablation catheter 200. The USB cable may be removed before use of the self-powered ablation catheter 200.

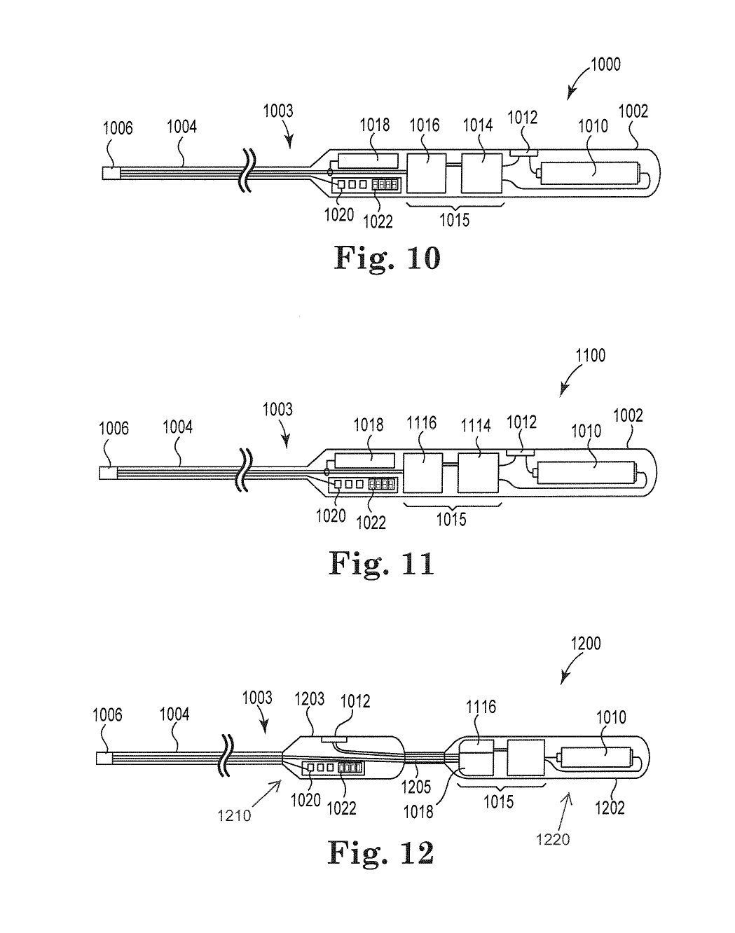

Referring now to FIGS. 10-12, there is illustrated various embodiments of a self-powered ablation catheter 1000, 1100, 1200 which include an ultrasound ablation arrangement in accordance with various embodiments of the disclosure. Although RF renal nerve ablation appears to be a viable approach, ultrasound renal nerve ablation provides more effective ablation with less artery wall injury. As an example, a cylindrical ultrasound transducer placed at the center of a renal artery produces a circumferential ring of ablated tissue. The arterial wall is spared from ablation by blood flow cooling. The catheter tip may contain a centering apparatus, such as a balloon or one or more centering baskets. The energy required for a circumferential ultrasound ablation is small enough to allow use of standard battery power. An ultrasound ablation system according to embodiments of the disclosure is faster and easier for clinicians to use when compared to RF approaches.

Conventional ultrasound consoles require separate ultrasound power generators and are cumbersome for clinicians, requiring a tethering connection to the catheter and connection to a wall electrical plug-in. Properly maintaining a sterile field is also needlessly complicated because of the tethering. Durable ultrasound consoles are typically a significant capital purchase by the customer, with additional cost and approvals. The console must be stored when not in use, and maintenance and calibration can be an issue. A self-contained power generator in the catheter handle, in accordance with various embodiments, eliminates the need for a tethering connection, power plug-in, capital purchase, maintenance, and storage hassles associated with conventional RF approaches.

According to some embodiments, it is desirable that the self-powered ablation catheters 1000, 1100, 1200 shown in FIGS. 10-12 be implemented as relatively low-cost devices, with at least the catheter portion of the devices being disposable. In some embodiments, it is desirable that the entire self-powered ablation catheter 1000, 1100, 1200 be implemented as low-cost and disposable devices. As will be described hereinbelow, attributes such as low-cost and disposability of the self-powered ablation catheters 1000, 1100, 1200 are largely achieved by implementing a design with power requirements that can readily be met using standard conventional or household batteries.

The embodiments shown in FIGS. 10-12 include a catheter 1003 and a handle unit 1001 coupled to the catheter 1003. The catheter 1003 includes a flexible shaft 1004 sufficient in length to access target tissue of a patient's body, such as a patient's renal artery or other tissue of the body. An ultrasound transducer 1006 is provided at a distal end of the shaft 1004 and coupled to the one or more electrical conductors that extend along the shaft 1004.

The handle unit 1001 includes a housing 1002 configured for hand-held manipulation. A battery 1010 and a power generator 1015 are each provided in the housing 1002. The battery 1010 is coupled to the power generator 1015 via a power switch 1012, which may be configured as an ON/OFF switch. The ON/OFF switch 1012 or a second ON/OFF switch can be implemented to activate and deactivate the power generator 1015. A control circuit 1018 is coupled to the battery 1010 and to one or more electrical conductors that extend from the handle unit 1002, along the catheter shaft 1004, and are coupled to the ultrasound transducer 1006. The control circuit 1018 includes a controller and memory that cooperate to control the various functions of the self-powered ablation catheter 1000, 1100, 1200. The control circuit 1018 may be configured to allow a clinician to adjust a limited number of operating parameters, such as choosing the cycle duration and duty cycle, for example. A user interface 1020/1022 can include various indicators and switches that facilitate clinician interaction with the ablation catheter 1000, 1100, 1200.

The self-powered ablation catheter 1000, 1100, 1200 can include one or more sensors, such as a temperature sensor to detect overheating of the ultrasound transducer 1006. Various displays 1020/1022 can be provided on the housing 1001 to indicate various types of information to the clinician. A sensor indicator display 1020 can be included on the housing 1001 and implemented to indicate various types of information and alerts, such as an over-temperature indication, fault situations, ON-OFF status, and proper operation indicators, for example. A timer display 1022 can be provided to show the duration of ablation or time remaining for an ablation procedure.

Various embodiments of a self-powered ultrasound ablation catheter 1000, 1100, 1200 may be configured for use with an external patient monitor of a type previously described, such as the patient monitor 110 shown in FIG. 4. A communications device, such as communications device 608 shown in FIG. 9, can be included in the handle electronics of the housing 1002, 1210. Selected components, features, and functions of the self-powered RF ablation catheters the patient monitor 110 discussed above can be incorporated in the context of various embodiments that include a self-powered ultrasound ablation catheter 1000, 1100, 1200 of the present disclosure.

The battery 1010 preferably serves as a sole source of power for the power generator 1015. More preferably, the battery 1010 serves as a sole source of power for all components of the self-powered ablation catheter 1000, 1100, 1200 that require power. The power generator 1015 and the ultrasound transducer 1006 are configured to generate ultrasound energy sufficient to ablate target tissue of the body using energy stored in the battery 1010.

Computer simulations conducted by the inventors indicate that the energy required for bilateral renal nerve ablation can easily be supplied by small conventional batteries. For example, it is been determined that the energy required for bilateral renal nerve ablation requires about 2,000 Joules. A conventional lithium AA battery is a capable of providing about 24,000 Joules, which easily meets the power requirements for bilateral renal nerve ablation of a self-powered ultrasound ablation catheter 1000, 1100, 1200 implemented in accordance with various embodiments. Advantageously, a ground pad and connection to a console is not required for conducting ultrasound ablation in accordance with embodiments of the disclosure.

Assuming about half the battery energy is wasted as transducer heat, computer simulations indicate that a circumferential ablation by ultrasound could be achieved using about 6 Watts. If a 2 minute ablation duration is required, about 720 Joules per renal artery, bilateral renal nerve ablation would take less than about 1,400 Joules. A battery capacity of 2,000 Joules would allow for extra capacity. Standard alkaline AA batteries, for example, hold about 12,000 Joules, and lithium AA batteries hold about 24,000 Joules, as mentioned previously. Because the power requirements of a self-powered ultrasound ablation catheter 1000, 1100, 1200 are so low, a variety of series and parallel battery arrangements can be used with inexpensive and readily available batteries to achieve the energy capacity, voltage, current draw, and storage life desired, with small weight and volume.

In the embodiments shown in FIG. 10, the power generator 1015 includes a step-up DC-to-DC converter 1014 that can be used to transform the low battery voltage into higher voltage to power the ultrasound transducer 1016. A simple oscillator circuit 1016 provides the needed frequency. Alternatively, and with reference to FIG. 11, an oscillator circuit 1114 is provided to convert the DC battery power to AC power. A conventional AC transformer 1116 can be used to step up the voltage to power the ultrasound transducer 1006. Other oscillator/transformer/converter arrangements can be used. Various hybrid arrangements can also be used.

In the embodiment shown in FIG. 12, a small catheter handle 1210 may be tethered a few inches to a control unit 1220 placed on a nearby table via a flexible tether 1205. The entire system 1200 is preferably permanently connected together and configured for single-use (disposable). A battery-powered tethered control unit similar to the control unit 1220 may be used, but with rechargeable or replaceable battery. Although the configuration shown in FIG. 12 eliminates the requirement for connecting to wall electrical power, connectors are required for the tether 1205, which introduces sterilization, storage, and maintenance issues. Accordingly, a completely disposable approach is preferred.

In accordance with some embodiments, an ultrasound ablation catheter configuration can include a motor-driven transducer rotation mechanism to facilitate multi-spot or circumferential ablation. For example, a known micromotor mechanism can be incorporated in the housing 1002, 1210 and coupled to the ultrasound transducer 1006 in accordance with various embodiments. Suitable micromotor mechanisms include those having small energy requirements that can be satisfied by the battery 1010 provided in the handle unit 1002, 1210, with an additional small control circuit. A motorized ultrasound ablation approach can also be used to incorporate ultrasound imaging to guide and assess the ablation. In this case, additional signal connections to a separate display may be required if a visual image is desired, which can be wired or wireless connections. Alternatively, ultrasound signals can be used to characterize tissue changes without an actual visual image display, with tissue changes being detected and a simple indicator light on the handle unit 1002, 1210 to indicate “successful ablation,” for example.

A self-powered ablation catheter 1000, 1100, 1200 can be delivered to target tissue of the body using a variety of techniques. According to some embodiments, a separate guiding catheter can be used to navigate through various vessels of the body to access the target tissue, such as a renal artery via the superior or inferior aorta. Ultrasound transducer 1006 and shaft 1004 are advanced through the guiding catheter and into the artery. A centering apparatus is activated, if applicable, and the ablation is performed. Alternatively, a steerable version of a self-powered ablation catheter 1000, 1100, 1200 can be advanced through a procedure introducer sheath (a short sheath that penetrates the skin and provides entry into the arterial system) and advanced through the arterial system and steered into and positioned within the renal artery for ablation. An over-the-wire technique can be used, with or without a guiding catheter, in which an ablation catheter 1000, 1100, 1200 is implemented to include a guidewire lumen extending from at least a proximal end of the catheter 1003 to the ultrasound transducer 1006, which may have a cylindrical shape with a central void through which the guidewire can pass. By way of example, a guidewire is advanced through the procedure introducer sheath to the target artery, and catheter 1000, 1100, 1200 is advanced into the target artery over the guidewire. A trocar may be used to access subcutaneous or abdominal target tissue, and the ultrasound transducer 1006 and shaft 104 of the ablation catheter 1000, 1100, 1200 can be advance to the target tissue. Other access approaches are contemplated.

Various self-power ultrasound ablation catheter embodiments can be constructed to include any of the features described in commonly owned U.S. Provisional Patent Application Ser. No. 61/491,728 filed on May 31, 2011, which is incorporated herein by reference. Various self-power ultrasound ablation catheter embodiments can be constructed to include any of the features described in commonly owned U.S. Provisional Patent Application Serial No. 13/086,116 filed on Apr. 13, 2011, which is incorporated herein by reference.

According to various embodiments, a self-powered ablation catheter can include an RF ablation arrangement and an ultrasound arrangement. In some embodiments, for example, the ultrasound arrangement is operated in a scanning or imaging mode, and the RF ablation arrangement is operated to ablate target tissue. The ultrasound arrangement can be used to locate suitable (e.g. non-diseased) target tissue, monitor progress of the ablation by scanning the target tissue during the procedure, and/or subsequently scan the ablated tissue to verify the efficacy of the ablation. In some embodiments, the RF ablation arrangement and an ultrasound ablation arrangement of a self-powered ablation catheter can be used for ablating target tissue, and the ultrasound arrangement can also be used for scanning or imaging. The RF and ultrasound ablation arrangements can be used in tandem or individually depending on the type of target tissue and environment of use.

Whereas conventional RF tissue ablation generators operate at a frequency near 500 kHz, and ultrasound ablation generators operate at frequencies above 1 MHz, a frequency in the range of 500 kHz to 10 MHz may be used for both RF and ultrasound ablation. According to some embodiments, a single ablation generator (e.g., a common ablation generator) is used to provide power to the ultrasound transducer and RF electrode in tandem (e.g., for concurrent operation) or individually (e.g., for selectable independent operation).

Since ultrasound transducers are typically coated with a good conductor, such as gold, to make electrical connection to the transducer, such a metalized surface (e.g., the outside surface of a cylindrical ultrasound transducer) may make contact with blood surrounding the transducer to provide a separate RF ablative path for current. In this mode, RF and ultrasound energy may be provided simultaneously through a single generator and a single ablation element to yield a desirable combination of the two ablation energies. The two energies typically have separate return electrodes. In other modes, the electrically conductive coating of an ultrasound transducer serves as an RF electrode and the ultrasound transducer is configured for scanning or imaging. In further modes, the electrically conductive coating of an ultrasound transducer serves as an RF electrode, and the ultrasound transducer is configured for ablating and scanning or imaging.

Various embodiments disclosed herein are generally described in the context of ablation of perivascular renal nerves for control of hypertension. It is understood, however, that embodiments of the disclosure have applicability in other contexts, such as performing ablation from within other vessels of the body, including other arteries, veins, and vasculature (e.g., cardiac and urinary vasculature and vessels), and other tissues of the body, including various organs. It is further understood that a self-powered RF or ultrasound ablation catheter of a type described herein can be implemented for cutaneous or subcutaneous applications, such as for ablating anomalous tissue on a patient's skin. Also, high frequency energy sources other than an RF generator may be used, such as a microwave generator.

It is to be understood that even though numerous characteristics of various embodiments have been set forth in the foregoing description, together with details of the structure and function of various embodiments, this detailed description is illustrative only, and changes may be made in detail, especially in matters of structure and arrangements of parts illustrated by the various embodiments to the full extent indicated by the broad general meaning of the terms in which the appended claims are expressed.

What is claimed is:

1. An apparatus, comprising:
a catheter, comprising:
a flexible shaft having a proximal end, a distal end, a length, and a lumen arrangement extending between the proximal and distal ends, the length of the shaft sufficient to access a patient's renal artery; and
an ablation element provided at the distal end of the shaft; and
a handle unit, comprising:
a housing configured for hand-held manipulation and coupled to the catheter;
a control circuit provided in the housing;
a battery provided in the housing; and
a generator provided in the housing and coupled to the battery and the control circuit, the generator configured to generate energy sufficient for the ablation element to ablate perivascular renal nerve tissue using only energy stored in the battery.

2. The apparatus of claim 1, wherein the battery comprises a battery volume no larger than that of an AA battery.

3. The apparatus of claim 1, wherein the battery comprises a battery volume no larger than two AA batteries.

4. The apparatus of claim 1, wherein the generator comprises a high frequency AC generator and the ablation element comprises at least one RF electrode.

5. The apparatus of claim 4, wherein the generator, powered solely by the battery, is configured to generate between about 8 Watts for up to about 2 minutes and about 30 Watts for up to 4 minutes for each of a patient's two renal arteries.

6. The apparatus of claim 4, wherein the generator, powered solely by the battery, is configured to generate between about 7,700 and about 12,000 Joules.

7. The apparatus of claim 4, wherein the generator, powered solely by the battery, is configured to generate between about 12,000 and about 30,000 Joules.

8. The apparatus of claim 4, comprising a cooling arrangement configured for providing cooling to the renal artery in proximity to the at least one RF electrode.

9. The apparatus of claim 1, wherein the generator comprises an ultrasound generator and the ablation element comprises an ultrasound transducer.

10. The apparatus of claim 9, wherein the battery is required to expend no more than about 2,000 Joules to denervate each of a patient's two renal arteries.

11. The apparatus of claim 9, wherein the battery is required to expend no more than about 1,400 Joules to denervate of each of a patient's two renal arteries.

12. The apparatus of claim 9, wherein the generator is situated in a secondary housing separate from the housing configured for hand-held manipulation and coupled thereto by a flexible tether.

13. The apparatus of claim 1, comprising a manipulatible switch arrangement and a display arrangement respectively supported by the housing.

14. The apparatus of claim 1, wherein one or both of the catheter and the handle unit are configured as disposable units.

15. The apparatus of claim 1, comprising a wireless communications device supported at least in part in the housing.

16. The apparatus of claim 1, comprising a communications device supported at least in part in the housing and a patient monitor coupled to a display, the communications device configured for effecting communication between the ablation apparatus and the patient monitor.

17. An apparatus, comprising:
a catheter, comprising:
a flexible shaft; and
an ablation element provided at a distal end of the shaft; and
a handle unit, comprising:
a housing configured for hand-held manipulation and coupled to the catheter;
a control circuit provided in the housing;
a battery provided in the housing; and
a generator provided in the housing and coupled to the battery and the control circuit, the generator configured to generate energy sufficient for the ablation element to ablate target tissue of the body using energy stored in the battery.

18. The apparatus of claim 17, wherein the generator comprises a high frequency AC generator and the ablation element comprises at least one RF electrode.

19. The apparatus of claim 17, wherein the generator comprises an ultrasound generator and the ablation element comprises an ultrasound transducer.

20. The apparatus of claim 17, wherein:
the generator comprises an RF generator and an ultrasound generator; and
the ablation element comprises at least one RF electrode and an ultrasound transducer, the at least one RF electrode coupled to the RF generator and the ultrasound transducer coupled to the ultrasound generator.

21. The apparatus of claim 17, wherein:
the generator comprises a single generator; and
the ablation element comprises at least one RF electrode and an ultrasound transducer, the at least one RF electrode and the ultrasound transducer respectively coupled to the single generator.

22. The apparatus of claim 17, wherein the ablation element comprises an ultrasound transducer comprising an electrically conductive coating, the coated ultrasound transducer serving as a combined RF and ultrasound ablation element.

23. A method, comprising:
supplying power using a battery provided in a hand-held self-powered handle unit of an ablation catheter device;
generating energy by a generator provided within the handle unit using power supplied by the battery, the battery serving as a sole source of power for the generator;
communicating the energy to an ablation element provided at a distal end of a catheter positioned in proximity to target tissue of a patient; and
ablating the target tissue using the energized ablation element.

24. The method of claim 23, wherein the energy is generated by a high frequency AC generator and the ablation element comprises at least one RF electrode.

25. The method of claim 23, wherein the energy is generated by an ultrasound generator and the ablation element comprises an ultrasound transducer.

26. The method of claim 23, wherein the energy is generated for an RF ablation element and an ultrasound transducer, the energy generated for the ultrasound transducer usable by the ultrasound transducer in one or both of an ablation mode and a scanning or imaging mode.
